SEQUENCE LISTING (6 sequences)

SEQ ID NO: 1 - DNA, 20 bp, artificial
  synthetic oligonucleotide for amplification of fungal rDNA
ttacgtccctgccctttgta

SEQ ID NO: 2 - DNA, 17 bp, artificial
  synthetic oligonucleotide for amplification of fungal rDNA
tcctgagggaaacttcg

SEQ ID NO: 3 - DNA, 24 bp, artificial
  synthetic oligonucleotide for sequencing fungal rDNA
gcatatcaataagcggaggaaaag

SEQ ID NO: 4 - DNA, 19 bp, artificial
  synthetic oligonucleotide for sequencing fungal rDNA
ggtccgtgtttcaagacgg

SEQ ID NO: 5 - DNA, 20 bp, artificial
  synthetic oligonucleotide for sequencing fungal rDNA
tcctccgcttattgatatgc

SEQ ID NO: 6 - DNA, 22 bp, artificial
  synthetic oligonucleotide for sequencing fungal rDNA
ggaagtaaaagtcgtaacaagg

(19) United States
(12) Patent Application Publication
(10) Pub. No.: US 2018/0265903 A1
(43) Pub. Date: Sep. 20, 2018

(54) PROCESS FOR THE PRODUCTION OF MALATE

(71) Applicant: B.R.A.I.N. AKTIENGESELLSCHAFT BIOTECHNOLOGY RESEARCH AND INFORMATION NETWORK AG, Zwingenberg (DE)

(72) Inventors: Lars M. Blank, Dortmund (DE); Nick Wierckx, Bemelen (NL); Thiemo Zambanini, Aachen (DE); Eda Sarikaya, Aachen (DE); Joerg Buescher, Pfungstadt (DE); Guido Meurer, Seeheim-Jugenheim (DE)

(21) Appl. No.: 15/537,598

(22) PCT Filed: Dec. 21, 2015
(86) PCT No.: PCT/IB2015/059817

§ 371 Date: Jun. 19, 2017

(30) Foreign Application Priority Data
Dec. 23, 2014 (EP) 14200111.4
May 15, 2015 (EP) 15167802.6

(51) Int. Cl. C12P 7/46; C12N 1/14

(52) U.S. Cl. CPC C12P 7/46; C12N 1/14

(57) ABSTRACT

The present invention relates to the microbial production of malate. Fungal cells belonging to a species of the family of Ustilaginaceae were found to be an excellent host for the production of malate from glycerol. The malate production rate of fungal cells belonging to a species of the family of Ustilaginaceae was increased significantly by laboratory evolution. After medium optimization, a cultivation with the evolved strain on minimal medium with glycerol, or mono- or disaccharides, like glucose or sucrose or oligosaccharides yields a high production rate of malate.

### FIELD OF THE INVENTION

The present invention relates to the microbial production of malate. Fungal cells belonging to a species of the family of Ustilaginaceae were found to be an excellent host for the production of malate from glycerol. The malate production rate of fungal cells belonging to a species of the family of Ustilaginaceae was increased significantly by laboratory evolution. After medium optimization, a cultivation with the evolved strain on minimal medium with glycerol, or mono- or disaccharides, like glucose or sucrose, or oligosaccharides yields in a high production rate of malate.

### BACKGROUND ART

Malic acid (MA) is an organic compound with the formula HO₂CCH₂CHOHCO₂H. It is a dicarboxylic acid that is made by all living organisms, contributes to the pleasantly sour taste of fruits, and is used as a food additive. Malic acid has two stereoisomeric forms (L- and D-enantiomers), wherein the L-isomer being the predominant form in nature. The salts and esters of malic acid are known as malates. The malate anion is an intermediate in the citric acid cycle.

L-Malic acid is the naturally occurring form, whereas a mixture of L- and D-malic acid is produced synthetically.

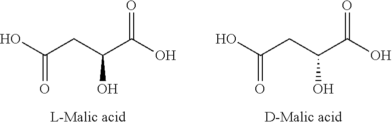 [SMILES: O=C(O)C[C@H](O)C(=O)O ; O=C(O)C[C@@H](O)C(=O)O]

Malic acid/malate plays an important role in biochemistry. In the C4 carbon fixation process, malate is a source of CO₂ in the Calvin cycle. In the citric acid cycle, L-malate is an intermediate, formed by the addition of an —OH group on the si face of fumarate. It can also be formed from pyruvate via anaplerotic reactions.

Malate is also synthesized by the carboxylation of phosphoenolpyruvate in the guard cells of plant leaves. Malate, as a double anion, often accompanies potassium cations during the uptake of solutes into the guard cells in order to maintain electrical balance in the cell. The accumulation of these solutes within the guard cell decreases the solute potential, allowing water to enter the cell and promote aperture of the stomata.

Biochemical evidence has been provided by Bais, H. P. et al. ((November 2008) Root-Secreted Malic Acid Recruits Beneficial Soil Bacteria Plant. Physiology, vol. 148, no. 3, 1547-1556), that the tricarboxylic acid cycle intermediate L-malic acid secreted from roots of Arabidopsis (Arabidopsis thaliana) selectively signals and recruits the beneficial rhizobacterium Bacillus subtilis FB17 in a dose-dependent manner. Root secretions of L-MA are induced by the foliar pathogen Pseudomonas syringae pv tomato (Pst DC3000) and elevated levels of L-MA promote binding and biofilm formation of FB17 on Arabidopsis roots. The demonstration that roots selectively secrete L-MA and effectively signal beneficial rhizobacteria establishes a regulatory role of root metabolites in recruitment of beneficial microbes, as well as underscores the breadth and sophistication of plant-microbial interactions. It was further demonstrated that malate is a second preferred carbon source for B. subtilis, which is rapidly co-utilized with glucose and strongly represses the uptake of alternative substrates (Sauer, U. et al., (Jan. 15, 2010) Metabolic Fluxes during Strong Carbon Catabolite Repression by Malate in Bacillus subtilis. The Journal of Biological Chemistry, 285, 1587-1596).

Malate has been identified by the US Department of Energy as one of the top 12 bio-based building blocks that can be converted into value added chemicals (Werpy, T. et al., (2004) Top value added chemicals from biomass. Volume 1-Results of screening for potential candidates from sugars and synthesis gas (No. DOE/GO-102004-1992). DEPARTMENT OF ENERGY WASHINGTON DC). Malate and its derivatives are also used as an acidulant in food products: malate=E296, NH₄-malate=E349, Na-malate=E350, K-malate=E351, Ca-malate=E352. The annual production is estimated between 10,000 tons (Sauer, M. et al., (2008) Microbial production of organic acids: expanding the markets. Trends Biotechnol. 26:100-108) and 40,000 tons (Goldberg, I. et al., (2006) Organic acids: old metabolites, new themes. J. Chem. Technol. Biotechnol. 81:1601-1611.). The projected market volume however is >200,000 tons (Sauer, M. et al., (2008) Microbial production of organic acids: expanding the markets. Trends Biotechnol. 26:100-108).

Currently malate can be produced by three routes on a large scale: chemical synthesis (a), enzymatic hydration of fumarate (b), fermentation (c). Industrial production mainly occurs via route (a), converting maleic anhydride catalytically to a racemic mixture (Brown, S. H. et al., (2012) Metabolic engineering of Aspergillus oryzoe NRRL 3488 for increased production of L-malic acid. Appl. Microbiol. Biotechnol. 97:8903-8912.).

In 1960 the first patent was filed on microbial malate production with Aspergillus flavus (Abe, S. et al., (1962) Method of producing L-malic acid by fermentation. U.S. Pat. No. 3,063,910). Over the years several other natural production hosts like Schizophyllum commune or Zygosaccharomyces rouxii have been found and optimized for the production of malate resulting in an Aspergillus oryzae overexpression mutant achieving a titer of 154 g/L malic acid with a rate of 0.94 g L⁻¹ h⁻¹ and a yield of 1.38 mol mol⁻¹ (69% of the theoretical maximum) (Battat, E. et al., (1990) Optimization of L-malic acid production by Aspergillus flavus in a stirred fermenter. Biotechnol. Bioeng. 37:11084116. Brown, S. H. et al., (2012) Metabolic engineering of Aspergillus oryzae NRRL 3488 for increased production of L-malic acid. Appl. Microbiol. Biotechnol. 97:8903-8912.). However all production processes start from glucose (Chen, X. et al., (2013) Metabolic engineering of Torulopsis glabrata for malate production. Metabol. Engin. 19:10-16.). Until today no microbial production process has been described converting glycerol to malate.

There are several disadvantages related to the existing production routes and processes (a), (b) and (c). Routes (a) and (b) are expensive since they require costly enzymes and reagents and rely on complex processes. Hence they are financially not suitable (Chen, X. et al., (2013) Metabolic engineering of Torulopsis glabrata for malate production. Metabol. Engin. 19:10-16.). The main drawback of route (c) is, that the best natural producer and most investigated organism for malate production Aspergillus flavus produces aflatoxins, making it not suitable for industrial scale production (Knuf, C. et al., (2013) Investigation of malate production in Aspergillus oryzae under nitrogen starvation conditions. Appl. Environ. Microbiol. 79(19):6050-6058.). Further, Aspergillus species are known to grow filamentously, resulting in problems with stirring and aeration during fermentation (Kuenz, A. et al., (2012) Microbial production of itaconic acid: developing a stable platform for high product concentrations. Appl. Microbiol. Biotechnol. 96(5):1209-1216. Gyamerah, M. H. (1995) Oxygen requirement and energy relations of itaconic acid fermentation by Aspergillus terreus NRRL 1960. Appl. Microbiol. Biotechnol. 44:20-26.).

Due to different drawbacks as indicated above, a considerable interest in fermentative production of malate has arisen.

The aim of the present invention is an improved process for the fermentative production of malate.

### SUMMARY OF THE INVENTION

The aim is achieved according to the invention by a method for the production of malate from glycerol, comprising culturing fungal cells belonging to a species of the family of Ustilaginaceae in a production medium containing glycerol, whereby malate is produced.

In one aspect of the present invention the species of the family of Ustilaginaceae is preferably a species of the genus of Ustilago and further preferred the species of the genus of Ustilago is Ustilago maydis or Ustilago trichophora.

In a further embodiment of the invention, the fungal cells are capable of producing malate from glycerol at a concentration of at least 10 g/L after 96 h in 50 mL shake flask production medium at 30° C., 200 rpm and 25 mm shaking diameter, initially containing 200 g/L glycerol and 100 g/L CaCO₃ in water, wherein the initial OD600 of the fungal cells capable of producing malate in the shake flask production medium is 0.3.

In another embodiment of the invention, the fungal cells belonging to a species of the family of Ustilaginaceae have the ability of the Ustilago triphophora cells deposited as NBRC 100155, NBRC 100156, NBRC 100157, NBRC 100158, NBRC 100159, NBRC 100160 or CBS 131473, or the Macalpinomyces mackinlayi cells deposited as BRIP 52549a, or the Sporisorium lanigeri cells deposited as BRIP 27609a, to produce malate from glycerol, after being cultured in a culture medium.

The culture medium of the invention comprises a carbon source, a nitrogen source, a phosphate source and a magnesium source.

The carbon source of the culture medium preferably is glycerol, the nitrogen source of the culture medium is preferably selected from one or more of ammonium chloride, ammonium sulfate, ammonium phosphate, sodium nitrate, ammonium nitrate, urea, yeast extract, and peptone, and more preferably is ammonium chloride, the magnesium source preferably is selected from one or more of MgSO₄ and MgCl₂ and the phosphate source preferably is selected from one or more of KH₂PO₄, H₃PO₄, K₂HPO₄, NaH₂PO₄ and Na₂HPO₄, and more preferably is KH₂PO₄.

The malate produced by the method of the present invention can be used for the preparation of a pharmaceutical, a cosmetic, a food, a feed or a chemical product.

A further aspect of the present invention is a fungal cell belonging to a species of the family of Ustilaginaceae and having the ability to produce malate from glycerol, wherein the fungal cells are capable of producing malate from glycerol at a concentration of at least 10 g/L after 96 h in 50 mL shake flask production medium at 30° C., 200 rpm and 25 mm shaking diameter, initially containing 200 g/L glycerol and 100 g/L CaCO₃ in water, wherein the initial OD600 of the fungal cells capable of producing malate in the shake flask production medium is 0.3.

A further aspect of the present invention is the use of fungal cells belonging to a species of the family of Ustilaginaceae for the production of malate from glycerol, wherein the fungal cells may preferably be defined as disclosed above.

A further aspect of the present invention is the use of fungal cells belonging to a species of the family of Ustilaginaceae for the production of malate from mono- or disaccharides, like glucose or sucrose, or oligosaccharides, wherein the fungal cells may preferably be defined as disclosed above.

A further aspect of the present invention is a production medium comprising fungal cells belonging to a species of the family of Ustilaginaceae, wherein it is preferred that the fungal cells are capable of producing malate from glycerol at a concentration of at least 10 g/L after 96 h in 50 mL shake flask production medium at 30° C., 200 rpm and 25 mm shaking diameter, initially containing 200 g/L glycerol and 100 g/L CaCO₃ in water, wherein the initial OD600 of the fungal cells capable of producing malate in the shake flask production medium is 0.3.

Another aspect from the present invention is a production medium comprising malate obtainable by a method as described above.

Malic acid obtainable by a method as described above is also an aspect of the present invention, wherein the malate contains detectable traces of the fungal cells belonging to a species of the family of Ustilaginaceae or their respective fungal DNA. The respective fungal DNA may be detected by procedures known in the art, for example by PCR.

### DETAILED DESCRIPTION OF THE INVENTION

The technical problem of the present invention, namely an improved process for the fermentative production of malate, is solved by the provision of the embodiments provided herein and as characterized in the claims.

In a first embodiment the invention relates to a method for the production of malate from glycerol, comprising culturing fungal cells belonging to a species of the family of Ustilaginaceae in a production medium containing glycerol, whereby malate is produced.

The term ‘malate’ as used herein is defined to include malic acid itself and all possible salts of malic acid. Preferably salts of malic acid encompassed by the present invention are commonly used salts of malic acid like Ca-malate, Na-malate, K-malate and NH₄-malate.

In one aspect of the present invention the species of the family of Ustilaginaceae is preferably a species of the genera of Ustilago, Macalpinomyces or Sporisorium and further preferred the species of the genus of Ustilago is Ustilago maydis or Ustilago trichophora, the species of the genus of Macalpinomyces is Macalpinomyces mackinlayi and the species of the genus of Sporisorium is Sporisorium lanigeri. The most preferred species of the present invention is Ustilago trichophora.

The malate produced by the method of the invention may be recovered from the production medium and optionally purified. The purification may be complete or partial and can be performed by methods known in the art, like centrifugation, re-crystallation, precipitation, solubilization, and chromatography.

Glycerol may be contained in the production medium in an amount of about 1 g/L to about 300 g/L, preferably in an amount of about 50 g/L to about 280 g/L, more preferably in an amount of about 100 g/L to about 250 g/L or even more preferably in an amount of about 150 g/L to about 220 g/L.

The glycerol may be purified glycerol (pure glycerol/pharma glycerol), partially purified glycerol or crude glycerol and can be the sole carbon source in the production medium of the method of the invention.

The term ‘glycerol’ as used herein can be used interchangeably with the terms ‘glycerin’, ‘glycerine’, ‘propanetriol’ and ‘1,2,3-trihydroxypropane’.

The term ‘purified glycerol’ (pure glycerol/pharma glycerol) as used herein is defined as purified, pure or pharma glycerol as available from commercial suppliers.

The term ‘crude glycerol’ as used herein is defined as any crude glycerol which can be for example obtained from industrial processes.

In another embodiment the carbon source of the production medium is one or more of mono- or disaccharides or oligosaccharides, preferably glucose or sucrose. The mono- and/or disaccharide is contained in the production medium in an amount of about 1 g/L to about 300 g/L, preferably in an amount of about 50 g/L to about 280 g/L, more preferably in an amount of about 100 g/L to about 250 g/L or even more preferably in an amount of about 150 g/L to about 220 g/L.

In one embodiment of the invention, the production medium does essentially not contain, preferably does not contain at least one of the essential nutrients for the growth of the fungal cells.

The essential nutrients for the growth of the fungal cells is defined in the present application as any nutrient other than the carbon or the oxygen source, which is essential for the growth of the fungal cells and may preferably be selected from the nitrogen source, the phosphate source, the magnesium source or the trace elements. Trace elements may be Zn, Mn, Co, Cu, Mo, Ca, Fe, B and/or K. More preferably the essential nutrient which is essentially absent in the production medium may be selected from the nitrogen source, the phosphate source or the magnesium source of the culture medium and more preferably the absent essential nutrient is the nitrogen source.

The terms ‘essentially not containing’, ‘essentially free’ or ‘essentially absent’ as used herein is defined to be an amount of an essential nutrient which is capable to start the malate production, for example a concentration of 0.5 g/L or below, preferably 0.1 g/L or below, more preferably 0.05 g/L or below, even more preferably 0.01 g/L or below, even more preferably 5 mg/L or below, most preferred 1 mg/L or below.

In a further embodiment of the invention, the production medium contains a pH-control system or a buffer, wherein when the production medium contains a buffer the buffer can be any suitable buffer and is preferably selected from CaCO₃, MES (2-(N-morpholino)ethanesulfonic acid) and H₃PO₄.

In one embodiment the pH-control system or the buffer retains the pH of the production medium in a range of about pH 3.0 to about pH 8.0, preferably about pH 5.0 to about pH 7.5, more preferably in a range of about pH 5.5 to about pH 7.0, and still more preferably the pH is in a range of about 5.8 to about pH 6.5 or the pH is retained to be 5.8 or 6.5.

The malate production by any method of the present invention can be performed at temperatures from about 25° C. to about 40° C., preferably at temperatures from about 27° C. to about 37° C., more preferably at temperatures from about 30° C. to about 35° C.

The use of CaCO₃ in the malate production by any method of the present invention can help to overcome potential product inhibition. The CaCO₃, used as buffer, precipitates with the produced malic acid forming solid calcium malate. As a consequence the malic acid is no longer dissolved in the medium and thus no longer inhibiting the cells. Potential product inhibition can therefore be overcome by the use of CaCO₃ in the malate production methods of the present invention.

In a further embodiment of the invention, the fungal cells are capable of producing malate from glycerol at a concentration of at least 10 g/L after 96 h in 50 mL shake flask production medium at 30° C., 200 rpm and 25 mm shaking diameter, initially containing 200 g/L glycerol and 100 g/L CaCO₃ in water, wherein the initial OD600 of the fungal cells capable of producing malate in the shake flask production medium is 0.3.

In another embodiment of the invention, the fungal cells belonging to a species of the family of Ustilaginaceae have the ability of the Ustilago triphophora cells deposited as NBRC 100155, NBRC 100156, NBRC 100157, NBRC 100158, NBRC 100159, NBRC 100160 or CBS 131473, or the Macalpinomyces mackinlayi cells deposited as BRIP 52549a, or the Sporisorium lanigeri cells deposited as BRIP 27609a, to produce malate from glycerol, after being cultured in a culture medium.

In still another embodiment the invention does not encompass unevolved species of the family of Ustilaginaceae deposited before the priority date of the present invention, like for example the Ustilago triphophora cells deposited as NBRC 100155, NBRC 100156, NBRC 100157, NBRC 100158, NBRC 100159, NBRC 100160 or CBS 131473, or the Macalpinomyces mackinlayi cells deposited as BRIP 52549a, or the Sporisorium lanigeri cells deposited as BRIP 27609a.

The terms ‘unevolved’ and/or ‘uncultured’ cells as used herein are defined to refer to cells which have not been cultured in a culture medium for many generations after the strain was isolated from nature. Thereby no selection took place among the variants of the cells that occur naturally by the inherent mutation of genomic information.

The fungal cells of the present invention may have been cultured for about 20 generations or more, like about 30, about 50, about 100, about 150, about 200 or about 300 generations or more in a culture medium as described herein, wherein in each generation step the fungal cells are grown to an OD600 value of about 2 to about 100, more preferably to an OD600 value of about 10 to about 50 and then diluted from about 1:2 to about 1:100000, more preferably from about 1:10 to about 1:1000 in new culture medium. Thereby, a selection for those variants of the cells that occur naturally by the inherent mutation of genomic information takes place that exhibit an increased growth rate and an improved glycerol metabolism.

After the evolution of the fungal cells of the present invention as described above, the fungal cells exhibit an faster growth behavior and/or an improved glycerol metabolism.

Additionally, after the evolution of the fungal cells of the present invention as described above, the fungal cells exhibit an faster growth behavior and/or an improved metabolism of mono- or disaccharides, like glucose or sucrose, or oligosaccharides.

The terms ‘the culturing’, ‘the culture’ and ‘ the evolution’ as well as the terms ‘to culture’ and ‘to evolve’ can be used interchangeably throughout the specification.

The OD600 values disclosed in the present application are measured in an Ultrospec 10 cell density meter (Amersham Biosciences, UK) in 4 mL Rotilabo polystyrol Makro cuvettes from Carl Roth (Karlsruhe, Germany) with distilled water as blank.

The culture medium of the invention comprises a carbon source, a nitrogen source, a phosphate source and a magnesium source.

The carbon source of the culture medium is preferably selected from one or more of glycerol, mono- or disaccharides, or oligosaccharides and more preferably is selected from glycerol, glucose or sucrose. And most preferably is glycerol. The nitrogen source of the culture medium is preferably selected from one or more of ammonium chloride, ammonium sulfate, ammonium phosphate, yeast extract and peptone, and more preferably is ammonium chloride. The magnesium source preferably is selected from one or more of MgSO₄ and MgCl₂. The phosphate source preferably is selected from one or more of KH₂PO₄, H₃PO₄, K₂HPO₄, NaH₂PO₄ and Na₂HPO₄, and more preferably is KH₂PO₄.

The culture medium of the invention may additionally contain trace elements, like for example Zn, Mn, Co, Cu, Mo, Ca, Fe, B and/or K, and vitamins, like for example L (D-)biotin, calcium D(+)pantothenate, nicotinic acid, myo-inositol, thiamine hydrochloride, pyridoxol hydrochloride, para-aminobenzoic acid.

The culture medium or the production medium of the present invention can additionally contain conventional additives known in the art, like for example defoamers. Suitable defoamers may be selected from methoxy propanol, decanol, imbentin-AGS/35, oleoyl-rac-glycerol, tween 20, tween 80, ucon HTF 14, antifoam A, mineral oil, paraffin oil, paraffin wax, rapeseed oil, brij 30, nonylphenyl-polyethylene glycol, nonylphenyl-polyethylenglycol-acetat, jemmanime M-600, polyethylen glycol 1000, polyethylene glycol 10000.

The culture medium as well as the production medium used in the present invention are artificial, not naturally occurring liquid aqueous media.

The carbon source in the culture medium or glycerol in the production medium of the invention, as well as the essential nutrients in both media can be added via the culturing or production time or be present in their whole amount at the beginning of the culturing/production.

The carbon source of the production medium of the invention may also be selected from mono- or disaccharides, like glucose or sucrose or from oligosaccharides and can be, as well as the essential nutrients, added via the production time or be present in their whole amount at the beginning of the production.

If the ingredients are encompassed in the culture medium in their whole amount at the beginning of the culturing the carbon source, preferably glycerol, is present in an amount of about 5 g/L or more, the nitrogen source, preferably ammonium chloride, is present in an amount of about 0.1 g/L or, the magnesium source, preferably MgSO₄×7H₂O, is present in an amount of about 0.01 g/L or more and the phosphate source, preferably KH₂PO, is present in an amount of about 0.1 g/L or more.

If the ingredients are added to the culture medium via the culturing time the carbon source, preferably glycerol, is present in an amount of at least about 1 g/L or more, and the nitrogen source, preferably ammonium chloride, is present in an amount of about 0.01 g/L or more, the magnesium source, preferably MgSO₄×7H₂O, is present in an amount of about 1 mg/L or more and the phosphate source, preferably KH₂PO, is present in an amount of about 0.01 g/L or more.

In one aspect of the invention, the fungal cells of the invention belonging to a species of the family of Ustilaginaceae produce malate from glycerol at a concentration of at least 10 g/L after 96 h in 50 mL shake flask production medium at 30° C., 200 rpm and 25 mm shaking diameter, initially containing 200 g/L glycerol and 100 g/L CaCO₃ in water, wherein the initial OD600 of the fungal cells capable of producing malate in the shake flask production medium is 0.3.

In the present invention the values for OD600 can be measured by procedures known in the art. The OD600 described in this application was measured in an Ultrospec 10 cell density meter (Amersham Biosciences, UK) in 4 mL Rotilabo polystyrol Makro cuvettes from Carl Roth (Karlsruhe, Germany) with distilled water as blank.

In one further aspect of the invention, the production medium can be the culture medium, wherein at least one of the essential nutrients for the growth of the fungal cells is essentially fully consumed by growth of the fungal cells.

The essential nutrients for the growth of the fungal cells is defined in the present application as any nutrient other than the carbon or the oxygen source, which is essential for the growth of the fungal cells and may preferably be selected from the nitrogen source, the phosphate source, the magnesium source or the trace elements. Trace elements may be Zn, Mn, Co, Cu, Mo, Ca, Fe, B and/or K. More preferably the essential nutrient which is essentially absent in the production medium may be selected from the nitrogen source, the phosphate source or the magnesium source of the culture medium and more preferably the absent essential nutrient is the nitrogen source.

The malate produced by any method of the present invention can be used for the preparation of a pharmaceutical, a cosmetic, a food, a feed or a chemical product.

A further aspect of the present invention is a fungal cell belonging to a species of the family of Ustilaginaceae and having the ability to produce malate from glycerol, wherein the fungal cells are capable of producing malate from glycerol at a concentration of at least 10 g/L after 96 h in 50 mL shake flask production medium at 30° C., 200 rpm and 25 mm shaking diameter, initially containing 200 g/L glycerol and 100 g/L CaCO₃ in water, wherein the initial OD600 of the fungal cells capable of producing malate in the shake flask production medium is 0.3.

The fugal cells of the invention have the ability to produce malate from glycerol, when being cultured in a production medium containing from about 1 g/L to about 300 g/L glycerol and which does essentially not, preferably does not contain at least one essential nutrient for the growth of the fungal cells.

The glycerol contained in the culture or production medium of the present invention may be replaced by one or more of mono- or disaccharides, preferably by glucose or sucrose or by oligosaccharides.

In a preferred embodiment of the invention the fungal cells are selected from Ustilago triphophora cells deposited as NBRC 100155, NBRC 100156, NBRC 100157, NBRC 100158, NBRC 100159, NBRC 100160 or CBS 131473, or from Macalpinomyces mackinlayi cells deposited as BRIP 52549a, or from Sporisorium lanigeri cells deposited as BRIP 27609a after being evolved like disclosed above.

The fungal cells belonging to a species of the family of Ustilaginaceae of the different embodiments of the invention can also comprise mutants or derivatives of a species of the family of Ustilaginaceae. Furthermore the fungal cells belonging to a species of the family of Ustilaginaceae of the different embodiments of the invention can also be genetically or metabolically engineered to improve the malate yield or productivity or to reduce by-product formations.

The term ‘genetically engineered’ is defined as the direct manipulation of the DNA contained in an organism using biotechnology. New DNA may be inserted in the host genome by first isolating and copying the genetic material of interest using molecular cloning methods to generate a DNA sequence, or by synthesizing the DNA, and then inserting this construct into the host organism. Genes may be removed, or “knocked out”, using a nuclease. Gene targeting is a different technique that uses homologous recombination to change an endogenous gene, and can be used to delete a gene, remove exons, add a gene, or introduce point mutations.

The term ‘metabolic engineering’ is defined as the practice of optimizing genetic and regulatory processes within cells to increase the cells' production of a certain substance by techniques as described above.

A further aspect of the present invention is the use of fungal cells belonging to a species of the family of Ustilaginaceae for the production of malate from glycerol, wherein the fungal cells may be as disclosed above.

Still a further aspect of the present invention is the use of fungal cells belonging to a species of the family of Ustilaginaceae for the production of malate from one or more of mono- or disaccharides, preferably of glucose or sucrose, or one or more of disaccharides, wherein the fungal cells may be as disclosed above. Preferably the fungal cells are cultured fungal cells as disclosed above.

The fungal cells belonging to a species of the family of Ustilaginaceae of the different embodiments of the invention may also be able to produce other dicarboxylic acids from glycerol. These other dicarboxylic acids and their salts may be selected from succinate, citrate and/or itaconate.

A further aspect of the present invention is a production medium comprising fungal cells belonging to a species of the family of Ustilaginaceae, wherein it is preferred that the fungal cells are capable of producing malate from glycerol at a concentration of at least 10 g/L after 96 h in 50 mL shake flask production medium at 30° C., 200 rpm and 25 mm shaking diameter, initially containing 200 g/L glycerol and 100 g/L CaCO₃ in water, wherein the initial OD600 of the fungal cells capable of producing malate in the shake flask production medium is 0.3.

It is further preferred that the production medium contains from about 1 g/L to about 300 g/L glycerol and does essentially not, preferably does not contain at least one essential nutrient for the growth of the fungal cells.

It is also further preferred that the production medium contains from about 1 g/L to about 300 g/L mono- and/or disaccharides, preferably glucose and/or sucrose or from about 1 g/L to about 300 g/L oligosaccharide(s) and does essentially not, preferably does not contain at least one essential nutrient for the growth of the fungal cells.

Another aspect of the present invention is a production medium comprising malate obtainable by a method described above.

Malate obtainable by a method as described above is also an aspect of the present invention, wherein the malate contains detectable traces of the fungal cells belonging to a species of the family of Ustilaginaceae or their respective fungal DNA.

The respective fungal DNA may be detected by procedures known in the art, for example by using PCR techniques for example as described in example 9 below.

The identification of species of the family of Ustilaginaceae as novel malate production hosts provides new possibilities to make a microbial production process for malate economically more feasible. The unicellular growth pattern of these organisms poses a significant advantage over other production strains. In addition, species of the family of Ustilaginaceae are non-toxic and grow readily on defined media. Furthermore the production process starts from glycerol, which is considered a waste product of biodiesel production. On top the production directly from crude glycerol without prior treatment is possible.

It must be noted that as used herein, the singular forms “a”, “an”, and “the”, include plural references unless the context clearly indicates otherwise. Thus, for example, reference to “a cell” includes one or more of such different cells.

As used herein, the conjunctive term “and/or” between multiple recited elements is understood as encompassing both individual and combined options. For instance, where two elements are conjoined by “and/or”, a first option refers to the applicability of the first element without the second. A second option refers to the applicability of the second element without the first. A third option refers to the applicability of the first and second elements together. Any one of these options is understood to fall within the meaning, and therefore satisfies the requirement of the term “and/or” as used herein. Concurrent applicability of more than one of the options is also understood to fall within the meaning, and therefore satisfies the requirement of the term “and/or” as used herein.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

As described herein, “preferred embodiment” means “preferred embodiment of the present invention”. Likewise, as described herein, “various embodiments” and “another embodiment” means “various embodiments of the present invention” and “another embodiment of the present invention”, respectively.

When used herein, the term “about” is understood to mean that there can be variation in the respective value or range (such as pH, concentration, percentage, molarity, time etc.) that can be up to 5%, up to 10%, up to 15% or up to and including 20% of the given value. For example, if a formulation comprises about 5 mg/mL of a compound, this is understood to mean that a formulation can have between 4 and 6 mg/mL, preferably between 4.25 and 5.75 mg/mL, more preferably between 4.5 and 5.5 mg/mL and even more preferably between 4.75 and 5.25 mg/mL, with the most preferred being 5 mg/mL. As used herein, an interval which is defined as “(from) X to Y” equates with an interval which is defined as “between X and Y”. Both intervals specifically include the upper limit and also the lower limit. This means that for example an interval of “5 mg/mL to 10 mg/mL” or “between 5 mg/mL and 10 mg/mL” includes a concentration of 5, 6, 7, 8, 9, and 10 mg/mL as well as any given intermediate value.

The invention encompasses the following items:

Item 1 is a method for the production of malate from glycerol, comprising culturing fungal cells belonging to a species of the family of Ustilaginaceae in a production medium containing glycerol, whereby malate is produced.

Item 2 is method of item, wherein the species of the family of Ustilaginaceae is preferably a species of the genera of Ustilago, Macalpinomyces or Sporisorium and wherein the species of the genus of the genera of Ustilago, Macalpinomyces or Sporisorium is more preferably Ustilago maydis, Macalpinomyces mackinlayi or Sporisorium lanigeri.

Item 3 is the method of any one of the preceding items, further comprising recovering malate from the production medium and optionally purifying the recovered malate.

Item 4 is the method of any one of the preceding items, wherein glycerol is contained in the production medium in an amount of about 1 g/L to about 300 g/L, preferably in an amount of about 50 g/L to 280 g/L, more preferably in an amount of about 100 g/L to about 250 g/L or even more preferably in an amount of about 150 g/L to about 220 g/L.

Item 5 is the method of any one of the preceding items, wherein glycerol is crude glycerol.

Item 6 is the method of any one of the preceding items, wherein glycerol is the sole carbon source.

Item 7 is the method of any one of the preceding items, wherein the production medium does essentially not contain, preferably does not contain at least one of the essential nutrients for the growth of the fungal cells.

Item 8 is the method of any one of the preceding items, wherein the production medium contains a pH-control system or a buffer, wherein then the production medium contains a buffer the buffer is preferably selected from CaCO₃, MES and H₃PO₄.

Item 9 is the method of any one of the preceding items, wherein the fungal cells are capable of producing malate from glycerol at a concentration of at least 10 g/L after 96 h in 50 mL shake flask production medium at 30° C., 200 rpm and 25 mm shaking diameter, initially containing 200 g/L glycerol and 100 g/L CaCO₃ in water, wherein the initial OD600 of the fungal cells capable of producing malate in the shake flask production medium is 0.3.

Item 10 is the method of any one of the preceding items, wherein the fungal cells belonging to a species of the family of Ustilaginaceae have the ability of the Ustilago triphophora cells deposited as NBRC 100155, NBRC 100156, NBRC 100157, NBRC 100158, NBRC 100159, NBRC 100160 or CBS 131473, or the Macalpinomyces mackinlayi cells deposited as BRIP 52549a, or the Sporisorium lanigeri cells deposited as BRIP 27609a, to produce malate from glycerol, after being cultured in a culture medium.

Item 11 is the method of item 10, wherein the culture medium contains a carbon source, a nitrogen source, a phosphate source, a magnesium source and trace elements, wherein preferably the carbon source is glycerol.

Item 12 is the method of item 10 or 11, wherein the fungal cells belonging to a species of the family of Ustilaginaceae produce malate at a concentration of at least 10 g/L after 96 h in 50 mL shake flask production medium at 30° C., 200 rpm and 25 mm shaking diameter, initially containing 200 g/L glycerol and 100 g/L CaCO₃ in water, wherein the initial OD600 of the fungal cells capable of producing malate in the shake flask production medium is 0.3.

Item 13 is the method of any one of the preceding items, wherein the production medium can be the culture medium, wherein at least one of the essential nutrients for the growth of the fungal cells is essentially fully consumed by growth of the fungal cells.

Item 14 is the method of any one of the preceding items, comprising further using the malate produced for the preparation of a pharmaceutical, cosmetic, food, feed or chemical product.

Item 15 are fungal cells belonging to a species of the family of Ustilaginaceae and having the ability to produce malate from glycerol, wherein the fungal cells are capable of producing malate from glycerol at a concentration of at least 10 g/L after 96 h in 50 mL shake flask production medium at 30° C., 200 rpm and 25 mm shaking diameter, initially containing 200 g/L glycerol and 100 g/L CaCO₃ in water, wherein the initial OD600 of the fungal cells capable of producing malate in the shake flask production medium is 0.3.

Item 16 is the use of a fungal cell according to item 15 for the production of malate from glycerol.

Item 17 is the use of a fungal cell according to item 15 for the production of malate from one or more of mono- or disaccharides, preferably glucose or sucrose or from one or more of oligosaccharides.

Item 18 is a production medium comprising fungal cells belonging to a species of the family of Ustilaginaceae, wherein the fungal cells are capable of producing malate from glycerol at a concentration of at least 10 g/L after 96 h in 50 mL shake flask production medium at 30° C., 200 rpm and 25 mm shaking diameter, initially containing 200 g/L glycerol and 100 g/L CaCO₃ in water, wherein the initial OD600 of the fungal cells capable of producing malate in the shake flask production medium is 0.3.

Item 19 is the production medium of item 18, wherein the production medium contains from about 1 g/L to about 300 g/L glycerol and does essentially not, preferably does not contain at least one of the essential nutrients for the growth of the fungal cells.

Item 20 is the production medium of item 18, wherein the production medium contains from about 1 g/L to about 300 g/L of one or more of mono- or disaccharides, preferably glucose or sucrose or from about 1 g/L to about 300 g/L of one or more of oligosaccharides and does essentially not, preferably does not contain at least one of the essential nutrients for the growth of the fungal cells.

Item 21 is the method of any one of the preceding items, the fungal cell of item 15 to 17, or the production medium of item 18 to 20, wherein the fungal cells belonging to a species of the family of Ustilaginaceae comprise mutants, derivatives, or genetically or metabolically engineered cells of a species of the family of Ustilaginaceae.

Item 22 is the method of items 7 or 13 or the production medium of item 19, wherein the essential nutrient is selected from nitrogen source, the phosphate source, the magnesium source or the trace elements.

Item 23 is a production medium comprising malate obtainable by the method according to any one of items 1 to 14.

Item 24 is malate obtainable by the method according to any one of items 1 to 14, wherein the malate contains detectable traces of the fungal cells belonging to a species of the family of Ustilaginaceae or their respective fungal DNA.

The invention is further illustrated by the Figures and Examples which are merely illustrative and are not constructed as a limitation of the scope of the present invention.

### DESCRIPTION OF THE FIGURES

FIG. 1: Laboratory evolution of Ustilago trichophora CBS 131473.

FIG. 2: Comparison of evolved (open symbols) and wild type (closed symbols) Ustilago trichophora CBS 131473 in CaCO₃ buffered mTm. Glycerol concentration (triangles), malate concentration (squares), OD600 (circles).

FIG. 3: Comparison of evolved (open symbols) and wild type (closed symbols) Ustilago trichophora CBS 131473 in MES buffered mTm. Glycerol concentration (triangles), malate concentration (squares), OD600 (circles).

FIG. 4: Comparison of wild type Ustilago trichophora in 100 g/L crude glycerol (open symbols) and pharma glycerol (closed symbols). Glycerol concentration (triangles), malate concentration (squares), OD600 (circles).

FIG. 5: Malate production for evolved Ustilago trichophora in 200 g/L crude glycerol and 100 g/L CaCO₃. Glycerol concentration (triangles), malate concentration (squares), OD600 (circles).

FIG. 6: Malate production for evolved Ustilago trichophora in 100 g/L crude glycerol and 100 mM MES. Glycerol concentration (triangles), malate concentration (squares), OD600 (circles).

FIG. 7: Malate production for evolved Ustilago trichophora in 100 g/L crude glycerol. Glycerol concentration (triangles), malate concentration (squares).

FIG. 8: Evolved Ustilago trichophora in bioreactor cultivation in production medium E containing 0.8 g/L NH₄Cl, 100 g/L glycerol and NaOH (used to keep the pH at 6.5). Glycerol concentration (triangles), malate concentration (squares), OD600 (circles).

FIG. 9: Evolved Ustilago trichophora in a fermentation in production medium E containing 1.6 g/L NH₄Cl, 100 g/L glycerol and NaOH (used to keep the pH at 6.5). Glycerol concentration (triangles), malate concentration (squares), OD600 (circles).

FIG. 10: Evolved Ustilago trichophora in a fermentation in production medium E containing 3.2 g/L NH₄Cl, 200 g/L glycerol and NaOH (used to keep the pH at 6.5). Glycerol concentration (triangles), malate concentration (squares), OD600 (circles).

FIG. 11: Evolved Ustilago trichophora in a fermentation in production medium E containing 3.2 g/L NH₄Cl, 200 g/L glycerol and 200 g/L CaCO₃ as buffer. Glycerol concentration (triangles), malate concentration (squares), OD600 (circles).

FIG. 12: Evolved Ustilago trichophora in a fermentation in production medium F containing 200 g/L CaCO₃. Glycerol concentration (triangles), malate concentration (squares), OD600 (circles).

FIG. 13: Concentrations of organic acids measured in the culture supernatants of a range of unevolved Ustilaginaceae after cultivation for 166.5 h in production medium E containing 0.8 g/L NH₄Cl, 100 g/L glycerol and 100 mM MES.

FIG. 14: Genomic context and primer positions of DNA amplification

FIG. 15: Malate concentration after 48 h in shake flask cultivations using glucose as sole carbon source. Ustilago trichophora CBS 131473 wild type and Ustilago trichophora CBS 131473 evolved towards improved growth as described in example 3 were compared.

FIG. 16: Fermentation of evolved Ustilago trichophora at different temperatures. A: OD600, B:malic acid concentration in fermentations at 25° C. (triangles), 30° C. (squares), 35° C. (circles) and cooling at temperatures exceeding 37° C. (diamonds). Error bars indicate deviation from the mean (n=2).

FIG. 17: Fermentation of evolved Ustilago trichophora at different pH-values. A: OD600, B: malic acid concentration in fermentations at pH 6.5 (squares), 5.5 (circles) and 4.5 (diamonds). Error bars indicate deviation from the mean (n=2).

FIG. 18: Fermentation of evolved Ustilago trichophora with CaCO3. A: Malic acid concentration (squares, solid line), glycerol concentration (triangles, dotted line), B: Fermentation broth after 264 h of fermentation.

It should be understood that the inventions disclosed herein are not limited to particular methodology, protocols, or reagents, as such can vary. The discussion and examples provided herein are presented for the purpose of describing particular embodiments only and are not intended to limit the scope of the present invention, which is defined solely by the claims. The following examples will illustrate the present invention.

### EXAMPLES

### Example 1: Cultivation and Multiplication of Ustilago

As standard growth medium, modified Tabuchi medium (mTm) containing 0.2 g/L MgSO₄×7H₂O, 10 mg/L FeSO₄×7H₂O, 0.5 g/L KH₂PO₄, 1 mL/L vitamin solution, 1 mL/L trace element solution and differing concentrations of NH₄Cl and glycerol was used.

Vitamin solution contained 0.05 g/L (D-)biotin, 1 g/L calcium D(+)pantothenate, 1 g/L 1 nicotinic acid, 25 g/L myo-inositol, 1 g/L thiamine hydrochloride, 1 g/L pyridoxol hydrochloride, 0.2 g/L para-aminobenzoic acid.

Trace element solution contained 15 g/L EDTA, 4.5 g/L ZnSO₄×7H₂O, 0.84 g/L MnCl₂×2H₂O, 0.3 g/L CoCl₂×6H₂O, 0.3 g/L CuSO₄×5H₂O, 0.4 g/L Na₂MoO₄×2H₂O, 4.5 g/L CaCl₂×2H₂O, 3 g/L FeSO₄×7 H₂O, 1 g/L H₃BO₃, 0.1 g/L KI.

As production medium A in shake flasks mTm containing 0.8 g/L NH₄Cl, 50, 100, 150, 200, 250 or 300 g/L glycerol and 100 g/L of CaCO₃ was used.

As production medium B in shake flasks 200 g/L glycerol and 100 g/L of CaCO₃ in deionized water was used.

As production medium C in shake flasks 100 g/L glycerol and 100 mM MES in deionized water was used.

As production medium D in shake flasks 100 g/L glycerol in deionized water was used without a buffer or pH-control system.

As production medium E in fermentations mTm containing differing concentrations of NH₄Cl, glycerol and buffer or a pH-control system was used.

As production medium F in fermentations double concentrated mTm containing 0.4 g/L MgSO₄×7 H₂O, 20 mg/L FeSO₄×7H₂O, 1 g/L KH₂PO₄, 2 mL/L vitamin solution, 2 mL/L trace element solution, 6.4 g/L NH₄Cl and 200 g/L glycerol was used.

As production medium G in shake flasks mTm containing 0.8 g/L NH₄Cl, 0 g/L glycerol, 100 g/L glucose, and 100 g/L of CaCO₃ was used. Of note, the production medium may also contain trace elements, vitamins and/or salts.

As preculture for production medium A 50 mL of mTm containing 0.8 g/L NH₄Cl, 50 g/L glycerol and 100 mM MES in a 500 mL shake flask were inoculated with 1 mL from a 24 h YEP culture containing 10 g/L peptone, 10 g/L yeast extract and 5 g/L NaCl. This culture was grown for 24 h and used to inoculate the production culture to a starting OD of 0.5.

As preculture for production medium G 50 mL of mTm containing 0.8 g/L NH₄Cl, 0 g/L glycerol, 50 g/L glucose and 100 mM MES in a 500 mL shake flask were inoculated with 1 mL from a 24 h YEP culture containing 10 g/L peptone, 10 g/L yeast extract and 5 g/L NaCl. This culture was grown for 14 h and used to inoculate the production culture to a starting OD of 0.5.

As preculture for production medium B, C, D, E, and F 50 mL of mTm containing 0.8 g/L NH₄Cl, 50 g/L glycerol and 100 mM MES in a 500 mL shake flask were inoculated with 1 mL from a 24 h YEP culture. This culture was grown for 24 h. The cells were transferred into the production medium B, C or D after washing twice by pelleting and resuspending the pellet in deionized water.

All shake flask cultures were incubated at 30° C. shaking at 200 rpm, with a shaking diameter of 25 mm and a relative air humidity of 80%.

### Example 2: Determination of Biomass

OD600 determination was performed in an Ultrospec 10 cell density meter (Amersham Biosciences, UK) in 4 mL Rotilabo polystyrol Makro cuvettes from Carl Roth (Karlsruhe, Germany) with distilled water as blank. Samples were diluted to an OD600 between 0.02 and 0.2 with distilled water. For each measurement, 3 mL of the diluted sample were placed in a fresh and clean cuvette. The cuvette was then placed in the Ultrospec 10 cell density meter, which was operated according to the manufacturer's instructions. For the calibration of OD600 measurement a linear range was found in which the R² value was above 0.999.

### Example 3: Adaptive Laboratory Evolution

For adaptive laboratory evolution, Ustilago trichophora CBS 131473 was grown in mTm as described in example 1 containing 0.8 g/L NH₄Cl, 100 mM MES and 50 g/L glycerol in 100 mL Erlenmeyer shake flasks with 10% filling volume incubated at 30° C. shaking at 200 rpm, with a shaking diameter of 25 mm and a relative air humidity of 80%. The first culture was inoculated to an OD600 of 0.5 from a 24 h YEP culture. OD600 was measured daily as described in example 2 until an OD600 of at least about 16 was reached. With this culture a new culture was inoculated to an OD600 of 0.5 sequentially for 57 days.

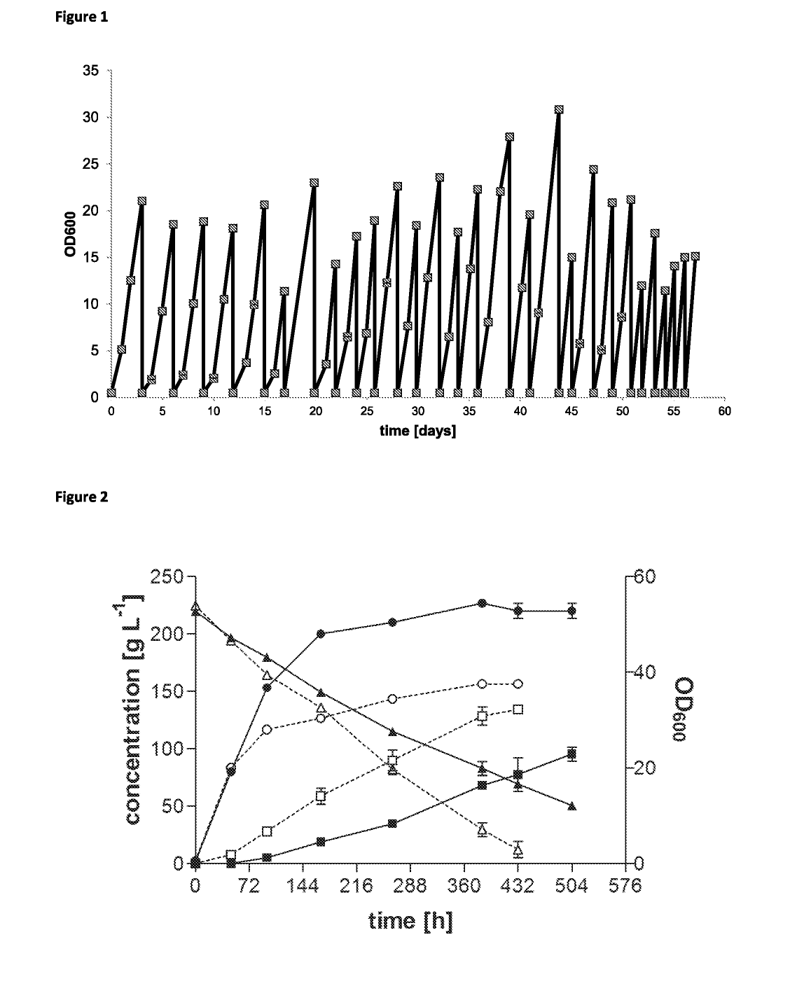

Since OD600 0.5 to 16 would be five generations, and there are 28 new inoculated cultures (FIG. 1) each (except for seven) grown to at least OD600 of 16, some even to OD600 of 32 (one generation more), the generation number performed is at least 140.

### Example 4: Quantification of Glycerol and Malate by HPLC

Quantification of malic acid was performed by ion exclusion high pressure liquid chromatography (IE-HPLC) using a 1200 series binary LC system and a 1200 series diode array detector (DAD) (Agilent Technologies, Waldbronn, Germany) and a RI2031Plus refractive index detector (RID) (Jasco, Gross-Umstadt, Germany). Ion exclusion separation was performed using an Aminex HPX-87H 300×7.8 mm column (Bio-Rad Laboratories, Munchen, Germany) and a KrudKatcher Classic inline filter (Phenomenex, Aschaffenburg, Germany). The mobile phase consisted of 30 mM sulfuric acid and 1% (v/v) acetonitrile in water (HPLC grade).

Whole culture samples were removed and 0.5 mL of sample were mixed with 0.2 mL of 37% hydrochloric acid. The samples where then filtered through a Chromfil Xtra H-PTFE-20/13 0.20 μm syringe filter (Macherey-Nagel, Düren, Germany) and 200 μL of the filtrates were placed into 96 well HPLC trays for acid and glycerol analysis.

IE-HPLC analysis was performed using an injection volume of 20 μL at a flow rate of 0.6 mL/min (isocratic) with a column temperature of 60° C. and a run time of 30 minutes. Detection was set at 210 nm, 8 nm band width. The quantitative capabilities of the ion exclusion method were determined for glycerol by performing replicate injections of serially diluted malic acid standards with concentrations ranging from 1.4-100 mM and by performing replicate injections of serially diluted glycerol standards with concentrations from 3.7-300 mM. The relative standard deviations for replicate injections was <5%. Malic acid and glycerol showed R²>0.99.

### Example 5: Malate Production

Ustilago trichophora CBS 131473, evolved as described in example 3, and Ustilago trichophora CBS 131473 wild type as reference were cultivated as described under example 1 in production medium A containing 0.8 g/L NH₄Cl and 200 g/L glycerol as sole carbon source or 0.8 g/L NH₄Cl and 50 g/L glycerol as sole carbon source.

The evolved strain consumes glycerol at a higher rate than the wild type strain (FIG. 2). The evolved strain also produces malate at a higher rate than the wild type strain. However, the OD600 observed for the evolved strain is about equal to that of the wild type strain at 48 h and lower at later time points. Taken together, these observations show that the evolved strain generates a higher product yield per substrate and can perform the conversion from glycerol to malate faster than the wild type strain.

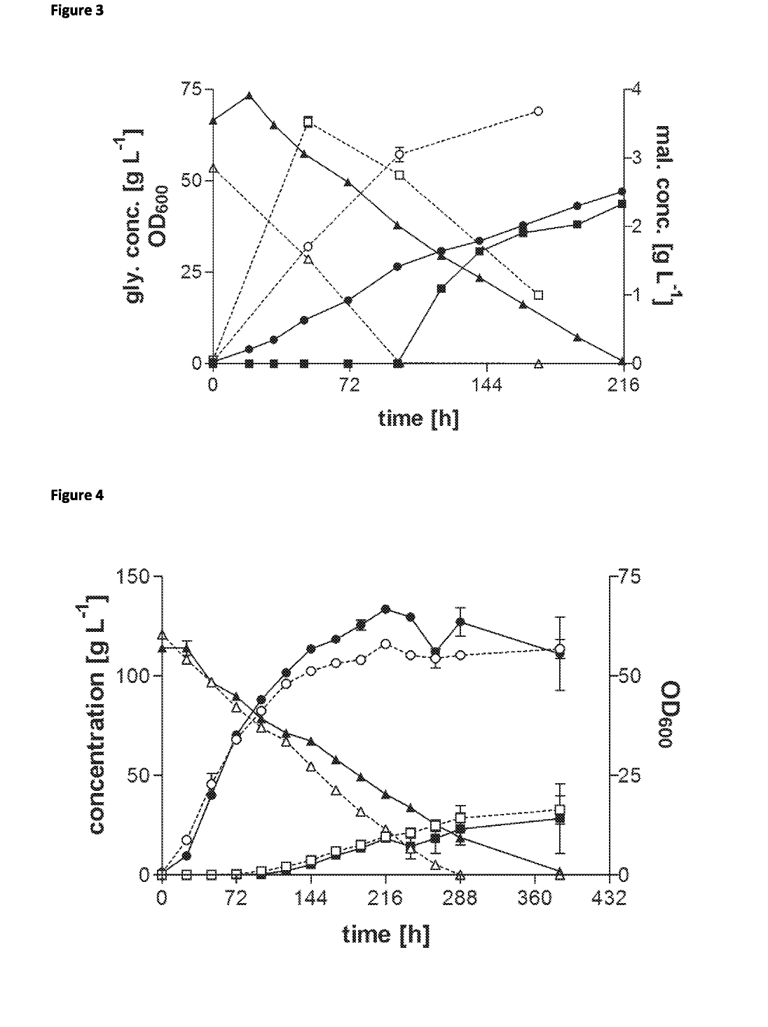

To better resolve the behavior of the strains in the initial 216 h, cultivations were performed at a lower initial glycerol concentration. FIG. 3 shows that the evolved strain grows faster in this initial phase of the cultivation, starts the production of malate faster than the evolved strain, and can produce malate at a higher rate (in the first 48 h). However, since glycerol becomes depleted during the cultivation of the evolved strain, this strain apparently starts to consume malate after glycerol depleted.

Ustilago trichophora CBS 131473 wild type was cultivated as described under example 1 in production medium A containing 0.8 g/L NH₄Cl and 100 g/L pharma/pure glycerol or crude glycerol as sole carbon source.

Overall, there is little difference in the behavior of the U. trichophora culture between pharma glycerol and crude glycerol (FIG. 4). The growth curve and the malate formation are highly similar; indeed the final OD600 and the final malate concentration are indistinguishable within the margin of the measurement error. The glycerol uptake rate even appears slightly higher when crude glycerol is used.

Ustilago trichophora CBS 131473, evolved as described in example 3, was cultivated as described under example 1 in production medium B.

This example demonstrates that a solution of a suitable concentration of glycerol that is buffered with a suitable buffer such as CaCO₃ suffices to enable the production of malate from glycerol. The OD600 increases despite the absence of several essential nutrients; presumably due to morphological changes and the formation of intracellular storage compounds.

Ustilago trichophora CBS 131473, evolved as described in example 3, was cultivated as described under example 1 in production medium C.

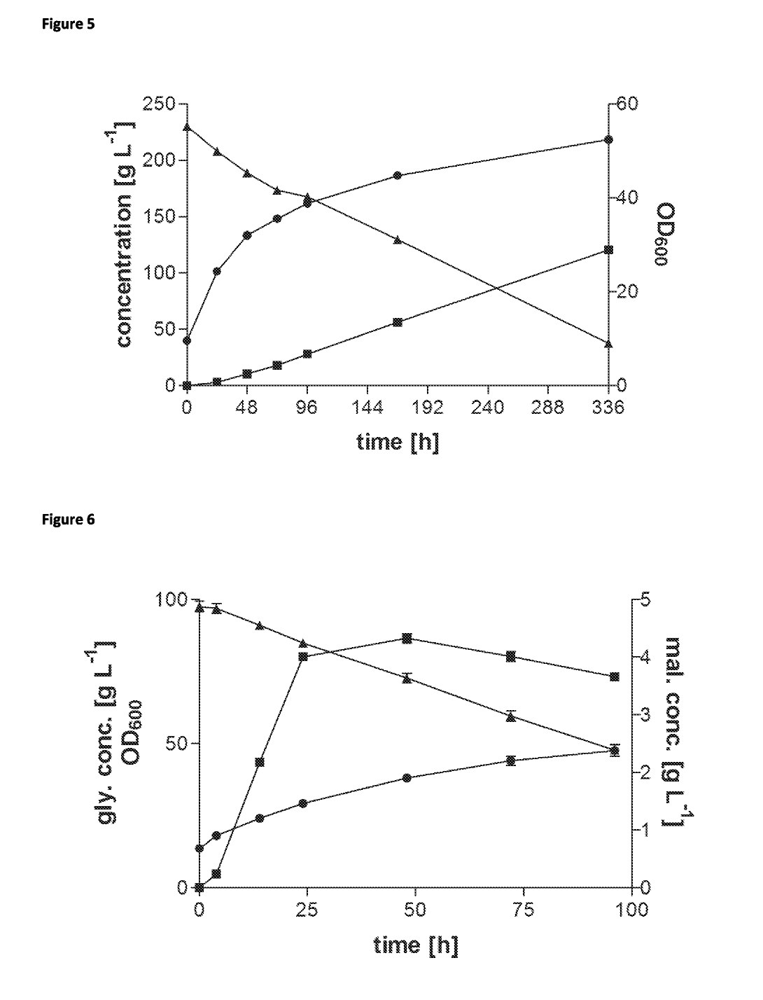

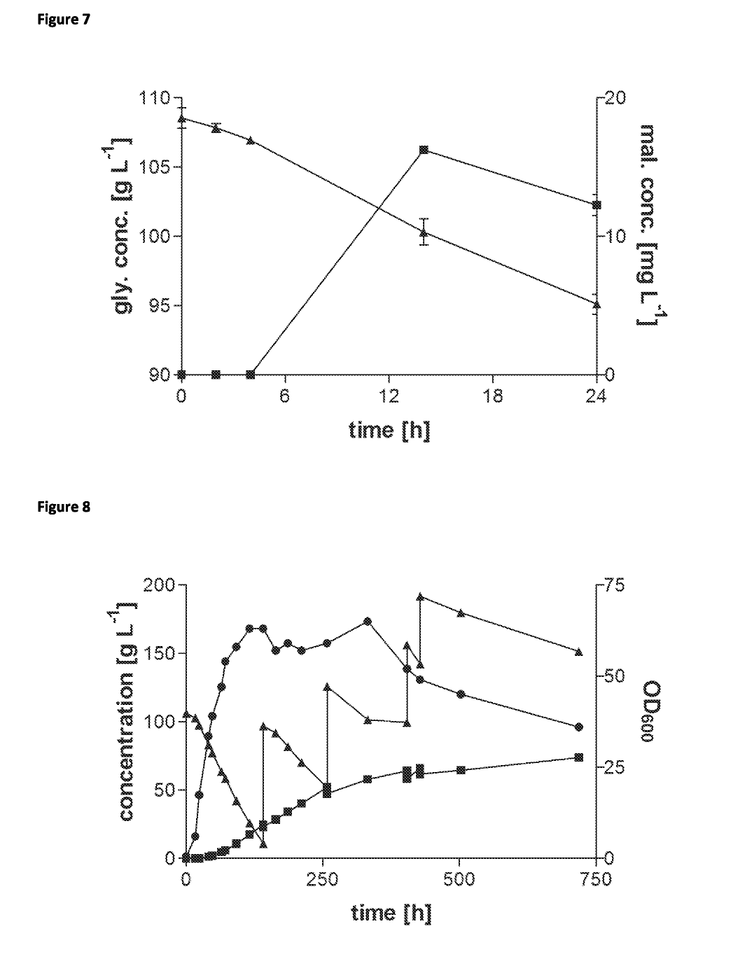

This example demonstrates that a solution of a suitable concentration of glycerol that is buffered with a suitable buffer such as MES suffices to enable the production of malate from glycerol (FIG. 6). Together with the data depicted in FIG. 5 this demonstrates that malate formation from glycerol does not depend on any particular buffer. However, since the buffer capacity of 100 mM MES is less than that of 100 g/L CaCO₃, malate formation stops earlier in FIG. 6 than it does in FIG. 5. Noticeably, glycerol consumption and OD600 increase continue even after malate production stopped.

Ustilago trichophora CBS 131473, evolved as described in example 3, was cultivated as described under example 1 in production medium D.

This example demonstrates that although malate production is even possible without buffer, only a very small amount of malate is produced if no buffer and no pH control system is used.

### Example 6: Production in Bioreactors

All batch cultivations were performed in New Brunswick BioFlo® 110 bioreactors (Eppendorf, Germany) with a total filling volume of 2.5 L and 1.25 L working volume. As medium mTm as described under example 1 was used. The temperature was maintained at 30° C. The pH was either set to 6.5 and controlled automatically with 10 M NaOH or different amounts of CaCO₃ were added as buffer. To prevent foam formation, antifoam 204 (Sigma Life Science, USA) was added upon level sensor control. The aeration rate was set to 1.25 L/min (1 m) and the dissolved oxygen tension (DOT) was kept at 80% saturation by automatically adjusting the stirring rate.

Ustilago trichophora CBS 131473, evolved as described in example 3, was cultivated as described under example 1 in production medium E containing differing amounts of NH₄Cl, 100 or 200 g/L glycerol as initial concentration and NaOH was used to keep the pH at 6.5.

FIG. 8 depicts that with 0.8 g/L NH₄Cl as the only available nitrogen source, the culture reaches an OD600 of about 65 and can maintain this cell density until about 2 weeks after the start of the cultivation. The initially available glycerol is almost completely consumed after about 1 week after the start of the cultivation. Subsequently, additional glycerol was added 4 times and malate production was sustained to reach a final concentration of about 60 g/L after about 740 h.

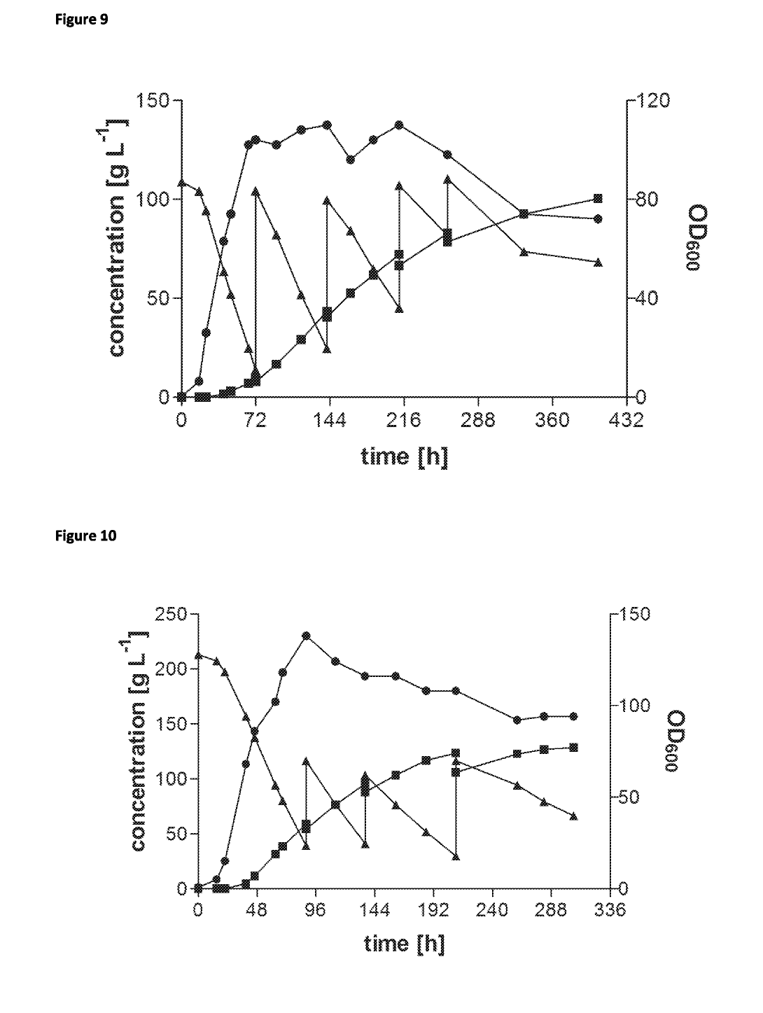

FIG. 9 depicts that with 1.6 g/L NH₄Cl as the only available nitrogen source, the culture reaches an OD600 of about 100 and thus about 50% higher than with 0.8 g/L NH₄Cl. The initially available glycerol is almost completely consumed within 72 h and thus at about twice the rate as in the cultivation depicted in FIG. 8. Moreover, additional glycerol was added 4 times and a high rate of glycerol consumption was sustained until about 300 h after the start of the cultivation. A final malate concentration of about 100 g/L was reached after about 400 h. Compared to the cultivation with 0.8 g/L NH₄Cl, the larger amount of cells in this cultivation can produce malate faster.

FIG. 10 depicts that with 3.2 g/L NH₄Cl as the only available nitrogen source, the culture reaches an OD600 of about 140 and thus about 40% higher than with 1.6 g/L NH₄Cl. To maintain the glycerol concentration above 10 g/L at all times during the cultivation, the initial glycerol concentration was 200 g/L and additional glycerol was added 3 times during the cultivation. A final malate concentration of about 120 g/L was reached after about 300 h. Compared to the cultivation with 0.8 g/L or 1.6 g/L NH₄Cl, the larger amount of cells in this cultivation can produce malate faster.

Ustilago trichophora CBS 131473, evolved as described in example 3, was cultivated as described under example 1 in production medium E containing 3.2 g/L NH₄Cl and medium F; both media containing 200 g/L glycerol as initial concentration and 200 g/L CaCO₃ as buffer.

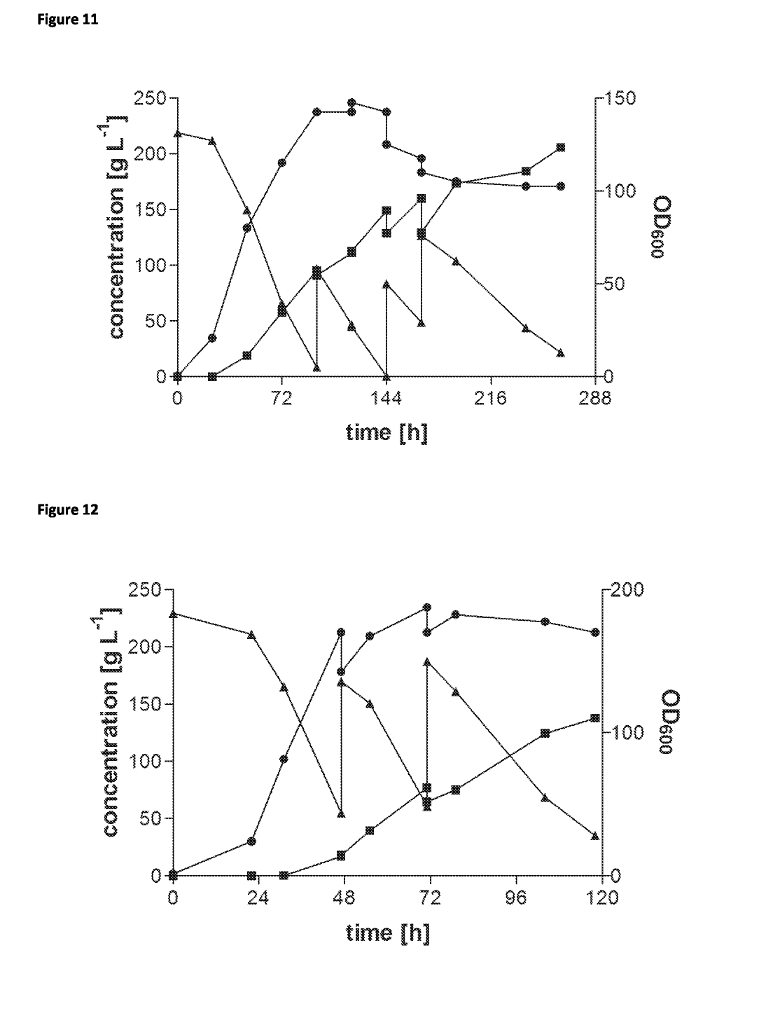

The cultivation depicted in FIG. 11 reaches an OD600 of about 150 after 4 to 5 days. The increased buffer concentration compared to the cultivation depicted in FIG. 10 permits the formation of about 200 g/L malate within 11 days. Additional glycerol was added to the culture to prevent glycerol limitation.

The cultivation depicted in FIG. 12 reaches about the same OD600 as the one depicted in FIG. 11, but in about half the time. This increased growth rate was enabled by doubled concentrations of all medium components except glycerol and CaCO₃. About 140 g/L of malate was produced in 120 h and thus at an average rate of greater than 1 g/L h.

### Example 7: Influence of Temperature

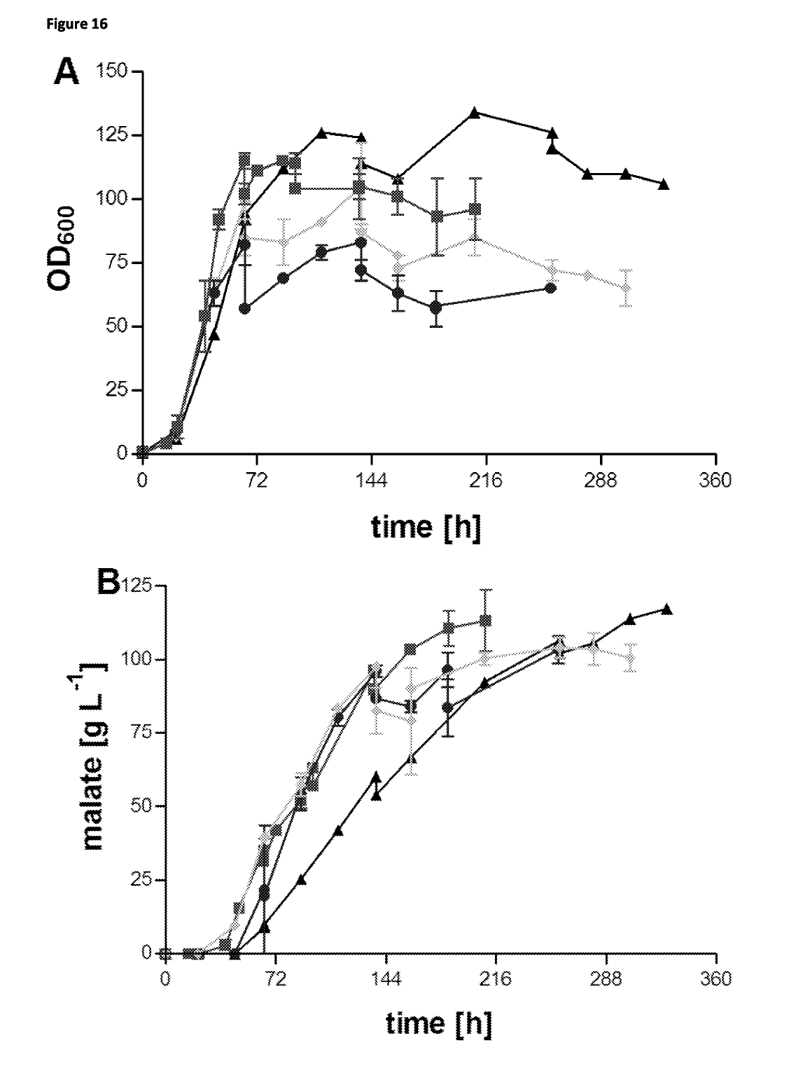

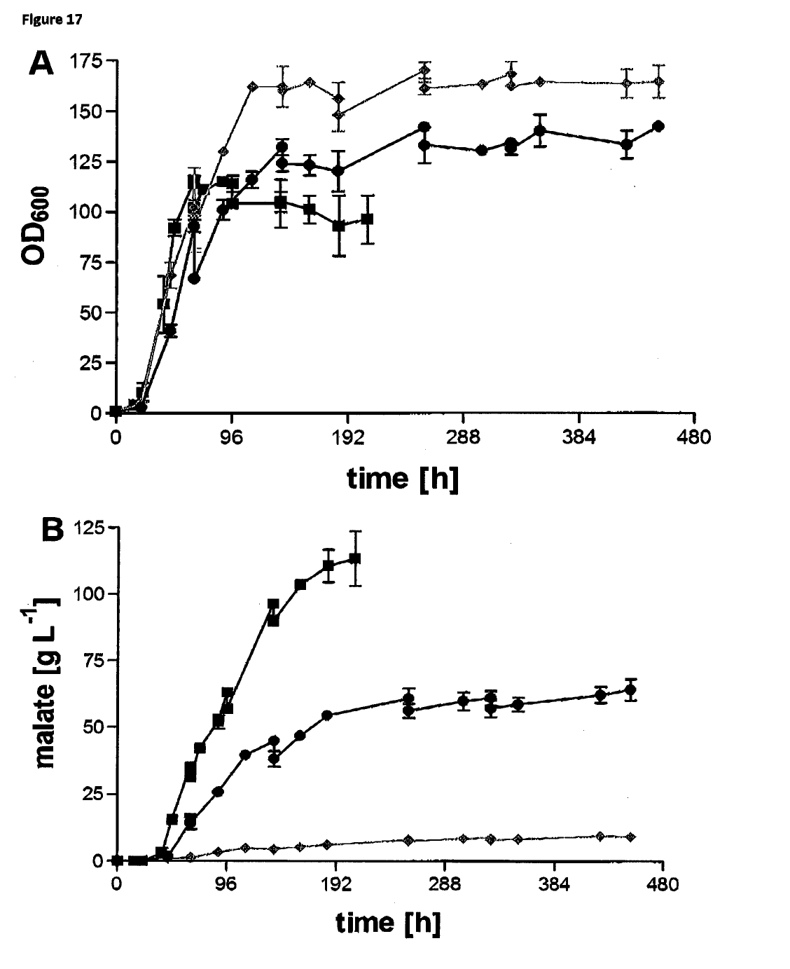

The influence of temperature was tested for malic acid production with Ustilago trichophora. To investigate influences of temperature on acid production and not cell growth, cells were grown at 30° C. and temperature was changed after growth phase to 25° C. and 35° C. In a third approach heating was disabled and cooling was only activated at temperatures exceeding 37° C. (FIG. 16). As seen in FIG. 17 B, malic acid production was not influenced by temperatures exceeding 30° C. However, 25° C. resulted in a slower malic acid production rate yet yielding the same final titer of around 120 g/L.

Since malic acid production with U. trichophora was not influenced by elevated temperatures and avoidance of heating and cooling system could reduce operating costs, two exemplary bioreactors were operated without heating and cooling system. While for one bioreactor neither growth nor malic acid production was affected, production rate and final titer in the second bioreactor decreased drastically, even though growth was not influenced. The reason for decreased acid production might be the temperature profile, which differed for the two bioreactors. While for bioreactor one temperature never exceeded 35.6° C., temperature for bioreactor two was above 37° C. for six hours of cultivation (29 h-35 h) reaching a maximum of 38.4° C. These data indicate that malic acid production with U. trichophora is temperature independent in a range between 30° C. and 37° C. Temperatures below 30° C. and temperatures exceeding 37° C. however seem to maleficently influence the process.

### Example 8: Influence of pH

Malic acid production with U. trichophora was investigated in bioreactors at pH 4.5, 5.5 and 6.5. pH did neither influence growth rate (FIG. 17A) nor morphology. However, maximal OD₆₀₀ was higher at lower pH. Further malic acid production was clearly lowered by decreasing pH reaching 113±15 g L⁻¹ (pH 6.5), 64±6 g L⁻¹ (pH 5.5) and 9±1 g L⁻¹ (pH 4.5).

### Example 9: Product Inhibition

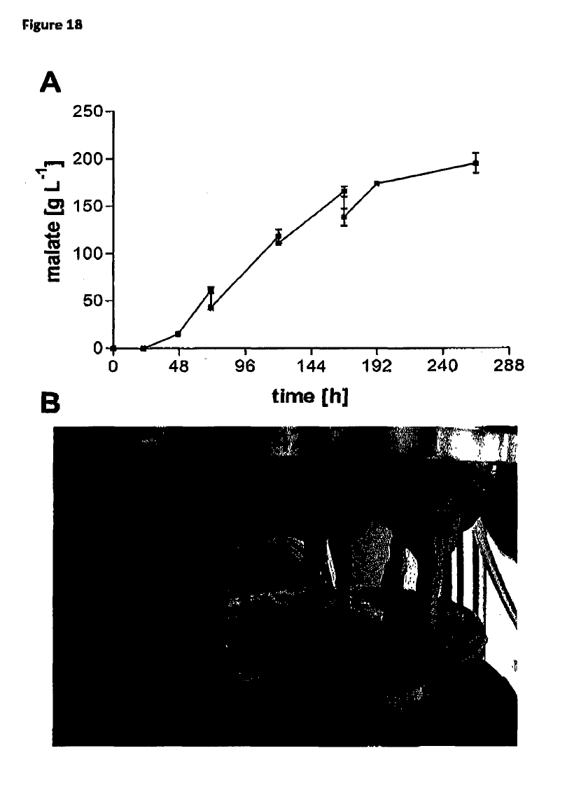

To overcome product inhibition in bioreactors, fed-batch bioreactor cultivation with MTM containing 3.2 g/L NH4Cl, 200 g/L glycerol and 100 g/L CaCO₃ as buffer was performed. Additional 100 g/L CaCO3 were added when pH dropped below 5.5. This fermentation resulted in 206 g/L of malic acid within 264 h of cultivation, corresponding to an overall production rate of 0.78 g L-1 h-1. The maximal production rate was 1.84 g/L (FIG. 18A). At this time point, fermentation had to be stopped, due to issues with medium viscosity (FIG. 18B). However, the prior limit of 140 g/L malic acid could be exceeded, further proving the hypothesis of product inhibition of concentrations above 140 g/L.

### Example 10: Detection of Fungal DNA in Malate

A sample of 2 μg of Ca-malate produced by a method of the present invention was dissolved in 1 μL of water as template in a polymerase chain reaction (PCR). Primers V9G and LR5 (table 3) are designed to amplify a part of any fungal chromosome that encodes the ribosomal RNA. With this pair of primers, a DNA fragment of about 1.8 kb is amplified by PCR, spanning the 18S, 5.8S and 28S genomic region (see FIG. 14). The constituents of the PCR reaction solution are listed in table 1. The temperature profile of the PCR reaction is listed in table 2.

TABLE 1
Ingredients of the 50 μl PCR reaction mix

| Component | Volume (Amount) | |
|---|---|---|
| Ca-malate (incl. template DNA) | 1 μL (2 mg/mL) | |
| Primer V9G | 1 μL (10 pmol) | |
| Primer LR5 | 1 μL (10 pmol) | |
| dNTPs | 1 | μL |
| 10x HotStarTaq buffer | 5 | μL |
| HotStarTaq polymerase | 0.5 | μL |
| ddH₂O | 40.5 | μL |

TABLE 2
PCR cycle program

| Step | Temp. [° C.] | Time [min] | Cycles |
|---|---|---|---|
| 1 | 95 | 15 | 1x |
| 2 | 95 | 0.5 | 30x |
| 3 | 58 | 0.5 | |
| 4 | 72 | 2 | |
| 5 | 72 | 10 | 1x |
| 6 | 10 | ∞ | 1x |

The product of the PCR reaction may be sequenced by a commercial sequencing service company using the NL-1, NL-4, ITS4, and ITS5 primers (table 3) to obtain 4 sequences. The sequences generated using the NL-1 and the NL-4 primers as well as the sequences generated using the ITS4 and ITS5 primers are aligned based on homology. The resulting contig of the ITS4 and ITS5 sequences is compared against the unite database (https://unite.ut.ee/). Based on similarity between the contig sequence and the sequences in the database, the species of the organism from which the contig sequence originates can be inferred as described by Nielsson et al, 2009: A software pipeline for processing and identification of fungal ITS sequences. Source Code for Biology and Medicine, 4 (1).

TABLE 3
Primers and their sequences

| Primer | Sequence 5′→3′ | |
|---|---|---|
| V9G (PCR) | TTACGTCCCTGCCCTTTGTA | Seq Id No: 1 |
| LR5 (PCR) | TCCTGAGGGAAACTTCG | Seq Id No: 2 |
| NL-1 (Seq) | GCATATCAATAAGCGGAGGAAAAG | Seq Id No: 3 |
| NL-4 (Seq) | GGTCCGTGTTTCAAGACGG | Seq Id No: 4 |
| ITS4 (Seq) | TCCTCCGCTTATTGATATGC | Seq Id No: 5 |
| ITS5 (Seq) | GGAAGTAAAAGTCGTAACAAGG | Seq Id No: 6 |

The resulting contig of the NL-1 and NL-4 sequences is compared against the NCBI nucleotide database using BLAST(http://blast.ncbi.nlm.nih.gov/Blast.cgi?PROGRAM=blastn&PAGE_TYPE=BlastSearch&LINK_LOC=blasthome).

Typically, only 10 cells per 5 μg of Ca-malate are required for a successful detection and identification (Ferrer et al 2001, Detection and Identification of Fungal Pathogens by PCR and by ITS2 and 5.8S Ribosomal DNA Typing in Ocular Infections, J. Clin. Microbiol, 39(8), 2873-2879).

### Example 11: Production of Dicarboxylic Acids from Glycerol

18 different strains belonging to the family of the Ustilaginaceae (Macalpinomyces mackinlayi BRIP 52549 a, Macalpinomyces ordensis BRIP 26904 a, Pseudozyma hubeiensis NBRC105054, Pseudozyma hubeiensis NBRC105055, Pseudozyma tsukubaensis NBRC 1940, Sporisorium cenchri-elymoidis BRIP 26491 a, Sporisorium iseilematis-ciliati BRIP 60887 a, Sporisorium lanigeri BRIP 27609 a, Ustilago trichophora NBRC100155, Ustilago trichophora NBRC100156, Ustilago trichophora NBRC100157, Ustilago trichophora NBRC100158, Ustilago trichophora NBRC100159, Ustilago trichophora NBRC100160, Ustilago trichophora CBS131473, Ustilago vetiveriae CBS131474, Ustilago xerochloae BRIP 60876 a, Ustilago xerochloae UMa702), were cultivated in a growth and production medium, containing 0.2 g/L MgSO₄×7H₂O, 10 mg/L FeSO₄×7H₂O, 0.5 g/L KH₂PO₄, 0.8 g/L NH₄Cl, 100 mM MES, 100 g/L glycerol, 1 mL/L vitamin solution, and 1 mL/L trace element solution.

Vitamin solution contained 0.05 g/L (D-)biotin, 1 g/L calcium D(+)pantothenate, 1 g/L 1 nicotinic acid, 25 g/L myo-inositol, 1 g/L thiamine hydrochloride, 1 g/L pyridoxol hydrochloride, 0.2 g/L para-aminobenzoic acid.

Trace element solution contained 15 g/L EDTA, 4.5 g/L ZnSO₄×7H₂O, 0.84 g/L MnCl₂×2H₂O, 0.3 g/L CoCl₂×6H₂O, 0.3 g/L CuSO₄×5H₂O, 0.4 g/L Na₂MoO₄×2H₂O, 4.5 g/L CaCl₂×2H₂O, 3 g/L FeSO₄×7 H₂O, 1 g/L H₃BO₃, 0.1 g/L KI.

As preculture the strains were grown in YEP medium containing 10 g/L peptone, 10 g/L yeast extract, and 5 g/L NaCl for 24 h.

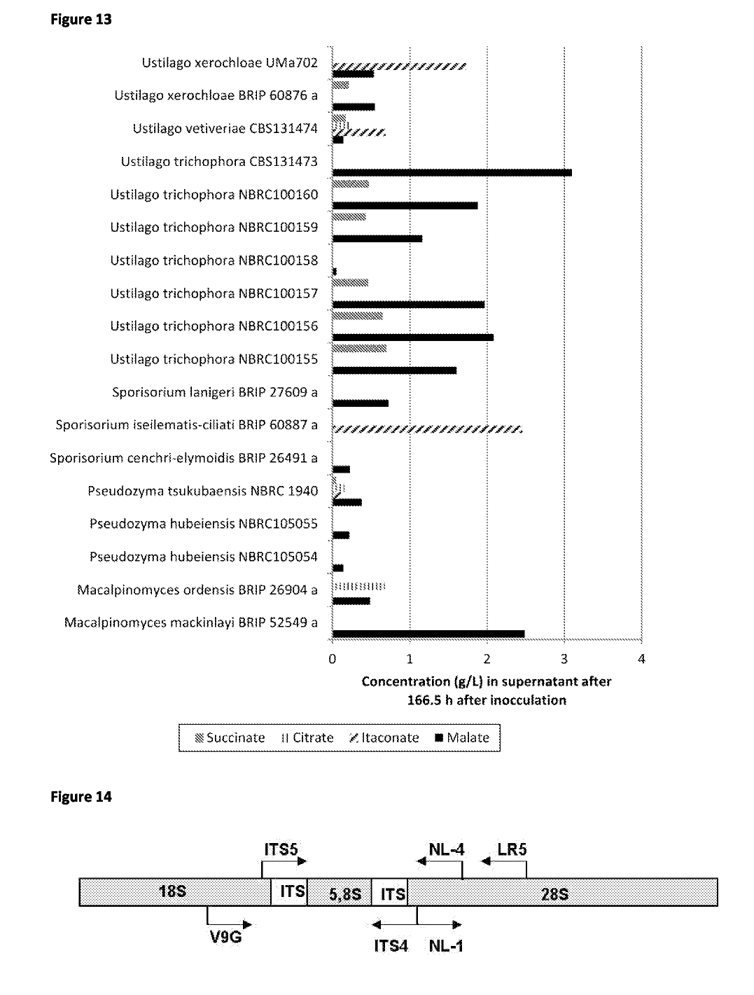

All cultivations were performed in 1.5 mL of medium in 24 deep well plates with square wells at 30° C. shaking at 300 rpm and with a shaking diameter of 50 mm. The main culture was inoculated from the preculture so that the initial OD600 of the main culture was 0.5. After 166.5 h of cultivation, the concentration of succinate, citrate, itaconate, and malate was determined as described in example 4. As depicted in FIG. 13, a wide range of strains belonging to the family of the Ustilaginaceae has the ability to form organic acids from glycerol. In particular, relevant concentrations of only malate and none of the other acids were observed in cultivations of Ustilago trichophora, Macalpinomyces mackinlayi, and Sporisorium lanigeri.

### Example 12

Ustilago trichophora CBS 131473, evolved as described in example 3, and Ustilago trichophora CBS 131473 wild type as reference were cultivated as described under example 1 in production medium G containing 0.8 g/L NH₄Cl, 0 g/L glycerol, and 100 g/L glucose as sole carbon source.

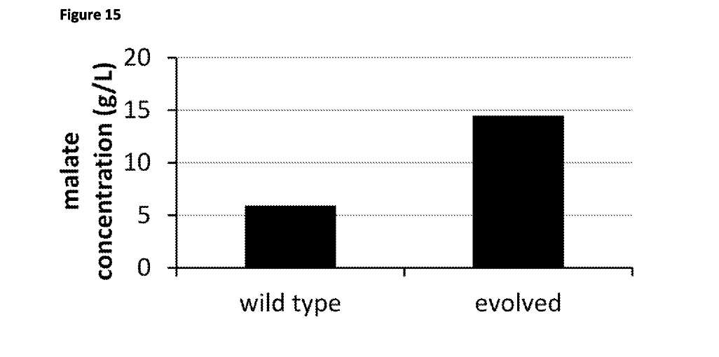

After 48 h, the evolved strain had produced almost three times as much malate as the wild type reference strain under the same conditions (FIG. 15). It should be noted that even though the strain was evolved using glycerol as the only carbon source, it also shows improved malate production when using a sugar as the only carbon source.

1.-17. (canceled)

18. A method for the production of malate from glycerol, comprising culturing fungal cells belonging to a species of the family of Ustilaginaceae in a production medium containing glycerol, whereby malate is produced.

19. The method of claim 18, wherein the species of the family of Ustilaginaceae is a species of the genera of Ustilago, Macalpinomyces or Sporisorium and wherein the species of the genus of the genera of Ustilago, Macalpinomyces or Sporisorium is Ustilago trichophora, Macalpinomyces mackinlayi or Sporisorium lanigeri.

20. The method of claim 18, wherein the glycerol is crude glycerol.

21. The method of claim 18, wherein the production medium contains a pH-control system or a buffer.

22. The method of claim 18, wherein the fungal cells are capable of producing malate from glycerol at a concentration of at least 10 g/L after 96 h in 50 mL shake flask production medium at 30° C., 200 rpm and 25 mm shaking diameter, initially containing 200 g/L glycerol and 100 g/L CaCO₃ in water, wherein the initial OD600 of the fungal cells capable of producing malate in the shake flask production medium is 0.3.

23. The method of claim 18, comprising further using the malate produced for the preparation of a pharmaceutical, cosmetic, food, feed or chemical product.

24. The method of claim 18, wherein the production medium contains from about 1 g/L to about 300 g/L glycerol and does not contain at least one of the essential nutrients for the growth of the fungal cells.

25. The method of claim 24, wherein the essential nutrient is selected from the nitrogen source, the phosphate source, the magnesium source or the trace elements.

26. Fungal cells belonging to a species of the family of Ustilaginaceae and having the ability to produce malate from glycerol, wherein the fungal cells are capable of producing malate from glycerol at a concentration of at least 10 g/L after 96 h in 50 mL shake flask production medium at 30° C., 200 rpm and 25 mm shaking diameter, initially containing 200 g/L glycerol and 100 g/L CaCO₃ in water, wherein the initial OD600 of the fungal cells capable of producing malate in the shake flask production medium is 0.3, wherein such fungal cells are obtainable by culturing the fungal cells for about 30 generations or more in a culture medium, wherein in each generation step the fungal cells are grown to an OD600 value of about 10 to about 50 and then diluted from about 1:10 to about 1:1000 in new culture medium.

27. The fungal cell of claim 26, wherein the fungal cells belonging to a species of the family of Ustilaginaceae comprise mutants, derivatives, or genetically or metabolically engineered cells of a species of the family of Ustilaginaceae.

28. A method for the production of malate from glycerol, comprising culturing the fungal cells of claim 26.

29. A method for the production of malate from one or more of mono- or disaccharides, comprising culturing the fungal cells of claim 26.

30. The method of claim 29, wherein the mono- or disaccharide is glucose or sucrose.

31. The method of claim 18, wherein the fungal cells belonging to a species of the family of Ustilaginaceae comprise mutants, derivatives, or genetically or metabolically engineered cells of a species of the family of Ustilaginaceae.

32. The method of claim 29, wherein the fungal cells belonging to a species of the family of Ustilaginaceae comprise mutants, derivatives, or genetically or metabolically engineered cells of a species of the family of Ustilaginaceae.

33. A production medium comprising fungal cells of claim 26.

34. The production medium of claim 33, wherein the production medium contains from about 1 g/L to about 300 g/L of one or more of mono- or disaccharides or contains from about 1 g/L to about 300 g/L of one or more of oligosaccharides, and does not contain at least one of the essential nutrients for the growth of the fungal cells.

35. The production medium of claim 34, wherein the production medium contains from about 1 g/L to about 300 g/L glycerol and does not contain at least one of the essential nutrients for the growth of the fungal cells.
